(19) United States
(12) Patent Application Publication
(10) Pub. No.: US 2026/0165875 A1
(43) Pub. Date: Jun. 18, 2026

(54) ORAL DEVICE FOR TREATING SLEEP APNOEA, CHRONIC SNORING OR OTHER CONDITIONS

(71) Applicant: AWE4 2015, S.L., Sant Cugat Del Valles (ES)

(72) Inventors: Joaquin SABARIS VILAS, Sant Gugat Del Valles (ES); Luis Amando GARCIA GONZALEZ, Nava (ES)

(21) Appl. No.: 19/127,055

(22) PCT Filed: Nov. 2, 2022
(86) PCT No.: PCT/ES2022/070704

§ 371 Date: May 2, 2025

(51) Int. Cl. A61F 5/56

(52) U.S. Cl. CPC A61F 5/566

(57) ABSTRACT

Particularly designed to prevent issues such as sleep apnea, snoring, bruxism, or other ailments. It is also useful for quick guidance or recognition of the patient's condition, in assisted cardiopulmonary resuscitation (CPR), cardioversion procedures, and for continuous remote patient monitoring. In this regard, the device comprises ferrous implants located in the meso-lingual area (beneath the tongue) and other regions of the soft palate. These serve as fixation means for the splint and a spring against vibrations of the surrounding tissue. The device includes a main body (1) based on an arched vertical septum, which terminates superiorly and inferiorly in two prosthetic bridges: a lower (2) and an upper (3). It also features sub-plates: an upper sub-plate (4) and a lower sub-plate (5), adapted to the user's dental arches. The lower prosthetic bridge (2) includes a magnetic element (7), while the upper includes a flexible arm (8) ending in a pair of adjustable terminals (9), intended to be positioned at the level of the palate. The flexible arm (8) acts as a shock absorber and/or retainer for surrounding structures at the tissue level.

### TECHNICAL FIELD

The present invention relates to a mouthguard with specific features that allow for the fixation of the tongue in an advanced position and mandibular retention at the positioning level. Additionally, it facilitates a PERMANENT CENTRAL AIRWAY ACCESS (air-breathing) and a structural retainer at the tissue level, which secures the mucosal layers of the palate and the surrounding muscles that, anatomically, may lead to an obstructive phase of the respiratory tract (apnea) and the vibration of these structures, resulting in chronic snoring.

The object of this invention is to provide a device capable of treating sleep apneas and chronic snoring, as well as being useful in other dental applications that may result from involuntary movement of the teeth and/or fatigue of the mandibular joint, such as bruxism, mandibular displacement, etc., and others, such as facilitating a patient's respiratory access during situations of CPR (cardiopulmonary resuscitation) or remote monitoring. This aims to reduce the use of positive pressure machines and external devices that may cause inconvenience to the patient, as well as dependence on external energy sources for operation.

### BACKGROUND OF THE INVENTION

Sleep apnea, or Obstructive Sleep Apnea Syndrome (OSAS), is a sleep disorder caused by intermittent cessation of breathing lasting more than 10 seconds due to an obstruction or collapse of the upper airway at the level of the hypopharynx or oropharynx; a potentially serious disorder that can impact the patient's health status. The initial precursor is often snoring, which can also lead to other consequences or repercussions.

This disorder affects up to 4-6% of the general population, especially overweight men, and can sometimes manifest with hypertension and coronary heart disease.

In its description, considering that during sleep a series of changes occur in the individual depending on alterations in muscle tone and the respiratory rate and rhythm, which can affect the act of breathing, and that the person is in an unconscious state, it is the nervous system that must maintain active the ventilatory muscles that keep the airway open. When there is a physical obstruction or some disorder in the coordination of the aforementioned system, obstructive sleep apnea occurs.
Obstructive sleep apnea, the most common form, occurs when the muscles of the throat relax, which, combined with poor mandibular positioning and postural issues, causes a discontinuity in breathing.
Central sleep apnea occurs when there is a decrease in the impulses to the muscles responsible for respiration.
Mixed sleep apnea syndrome occurs when both types are present.

Among the various factors that can lead to OSAS are anatomical factors such as:
Lingual hypertrophy (enlarged tongue)
Tonsillar hypertrophy
Cranio-mandibular malformations
Obesity
Postural disorders

Other habits that contribute to or exacerbate this syndrome (OSAS) include alcohol consumption, smoking, cranio-cervical alterations, use of central nervous system depressants, acromegaly, and hypothyroidism, with the latter being among the most common.

The most frequent signs of OSAS include nocturnal snoring and daytime sleepiness, although they can also be accompanied by:
Insomnia
Sweating
Enuresis
Gastroesophageal reflux
Seizures
Sleep interruption

The daytime symptoms associated with hypersomnolence include:
Headaches
Decreased libido
Impotence
Psychiatric disorders
Dry mouth
Voice alterations
Memory deficits
Intellectual deterioration
Irritability
Among others . . . .

In summary, it is the most common sleep disorder (along with insomnia), characterized by frequent awakenings during the night, snoring, and other issues or inconveniences. It is diagnosed through a sleep study or polysomnography, and treatment involves various medical disciplines due to its neurological, anatomical, ventilatory, and dental implications.

Regarding existing treatments to date, the following are noteworthy:
Hygienic-dietary measures
Weight loss as the primary factor to correct
Reducing alcohol consumption
Eliminating stimulants such as caffeine
Sleep hygiene practices
Avoiding relaxing medications at night
Postural adjustments during sleep
Regular exercise
CPAP systems (Continuous Positive Airway Pressure)

Primarily for moderate to severe cases, although with physical limitations, involving a portable system that applies positive, continuous pressure to the airway via the nose and mouth during sleep. Its use should be indefinite, as it is not a curative treatment.

### Surgical Procedures

To correct specific anatomical abnormalities affecting the upper airway, such as enlarged tonsils and nasal obstructions. When no obvious anatomical abnormalities are present, other surgical options include correction of the uvula and palate (uvulopalatopharyngoplasty) or mandibular adjustments.

### Pharmacological

Drugs such as progesterone, amitriptyline, theophylline, or oxygen therapy.

Finally, it should be noted that there are oral devices made up of splints, which always depend on the prior implantation of multiple implants in the patient, as can be seen, for example, in patent US 2004049102 by Lionel M. Nelson, which complicates their implantation.

### BRIEF DESCRIPTION OF DRAWINGS

To complement the subsequent description and to aid in a better understanding of the features of the invention, according to a preferred embodiment of the same, an illustrative, non-limiting drawing is included as an integral part of this description, which depicts the following:

FIG. 1: Shows a perspective view of a mouth device for treating sleep apnea and chronic snoring, made in accordance with the object of this invention.

FIG. 2: Shows a perspective view of the aforementioned mouth device, incorporating a mouthpiece with additional holes on the sides of the arched frontal septum to release internal pressure and/or to prevent noise during breathing and/or to avoid accumulation of debris that could lead to mycosis.

FIG. 3: Shows a perspective view of the same mouth device as in FIG. 2, but from the opposite side for better understanding.

### PREFERRED EMBODIMENT OF THE INVENTION

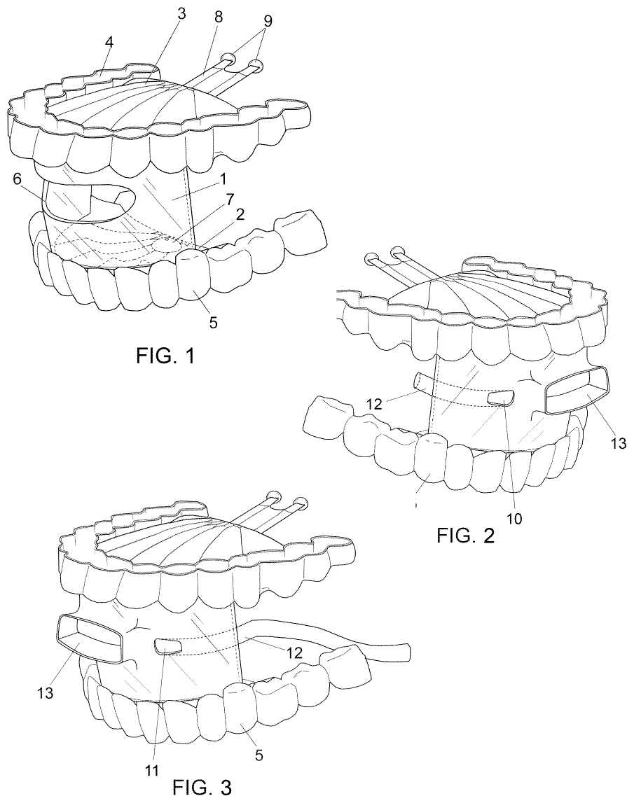

Referring to FIG. 1, it can be observed that the mouth device for treating sleep apnea and chronic snoring of the invention is constituted by a type of dental splint, which includes a main body (1) based on an arched vertical septum. This body terminates superiorly and inferiorly in two prosthetic bridges: a lower (2) and an upper (3), designed to adapt to the palate area and the sub-lingual area of the user. The body is further finished at its top and bottom with respective sub-plates: an upper sub-plate (4) and a lower sub-plate (5), adapted to the dental arches of the user.

According to one of the essential features of the invention, it has been foreseen that optionally, the device can be complemented with ferrous implants that must be previously implanted in the patient—one located along the longitudinal axis of the lower lingual region and another series at the level of the palate. The lower prosthetic bridge (2) includes a complementary magnetic element (7) that interacts with the implant located in the lower tongue area described above, stabilizing the device relative to tongue movement within the oral cavity.

Meanwhile, the upper prosthetic bridge (3) features at its posterior end a flexible arm (8), which terminates in a pair of terminals (9). These terminals can be magnetized and are spherical or otherwise adaptable in shape. They are positioned in a regulated manner on the palate—either without prior implants or with the aforementioned ferrous implants—allowing, during operation, for the extension arm (8) to act as a shock absorber and/or retainer for surrounding tissue structures at a tissue level.

As previously mentioned, the flexible arm allows for the attachment of two balls or terminals (9) at different angles, which can be easily varied in position to support the tissue of the palate, without the need for additional implants in this upper area. According to another feature of the invention, to enable breathing through the device, the main body (1) will include a wide front window (6), which can also be used in emergency situations for performing cardiopulmonary resuscitation (CPR). The device includes means for color change detection via sensor or pre-identification based on the patient's initial condition. Additionally, the device is capable of incorporating a sensor that monitors the vibration state of the palate as an initial condition and/or after treatment, providing new adjustment points or calibration for the extension arm (8) functioning as a shock absorber, for calibration and/or repositioning. Other sensors that could be integrated into the invention include flow meters and/or accelerometers or other types such as temperature sensors or blood oxygenation sensors, for example, luminescent sensors, etc. All these sensors will be controlled through a software application or app, which analyzes all received information.

In FIG. 2, it can be seen that the front window is realized as a tubular mouthpiece (13) that allows for the attachment of orotracheal cannulas or other devices. It also prevents the main air opening for the user from closing due to lip closure. Additionally, it incorporates a supplementary safety hole (10) in the main body (1) for air intake in case the front window becomes obstructed. This hole (10) includes a conduit (12) designed to connect to deep throat cannulas that extend toward the back of the throat, allowing for extra pressure relief and reducing friction noises caused by breathing through a conduit. It also helps eliminate extra surfaces that are difficult to access and could potentially develop fungal areas, such as candidiasis.

In FIG. 3, a perspective view from the opposite side shows another hole (11), which provides an air entry point via a tube (12) with similar characteristics to the previous one. Lastly, it should be noted that the described device could optionally incorporate sensors such as airflow measurement sensors, heart rate sensors, oxygen saturation sensors, or others. These measurements can be monitored through a software application installed on a mobile phone, tablet, or similar device, which would include the necessary electronics for control and data transmission.

### DETAILED DESCRIPTION OF THE INVENTION

The oral device for the treatment of sleep apnea, chronic snoring, and other related conditions, proposed by this invention, addresses the aforementioned problem by constituting a personalized mouthguard that fits within the oral cavity and adapts to various upper and lower dental arches. It allows for the fixation of the tongue through the magnetic attachment of a single prior implant positioned longitudinally in the mid-lower part of the tongue. Additionally, it includes a prosthetic bridge at the palate level, which is accompanied by an adjustable extension arm with terminals that can be magnetized at the tip, in the form of a ball, positioned in a regulated manner over the palate.

Optionally, and only if necessary, ferrous implants could be previously placed at the palate level, allowing the extension arm to act as a shock absorber and/or retainer of the surrounding tissue structures, due to the flexible nature of this arm.

In this case, the repeatedly mentioned arm has a flexible nature, making it adjustable. It allows the two balls to be held at variable angles to support the palatal tissue, without the need for additional implants beyond the sublingual implant.

Consequently, the sublingual implant is the only prior implant used to fix the tongue in place.

Thanks to this, the elimination of snoring and the clearance of the airways are supported by securing the respiratory tract and reducing internal air pressure or resonance.

Each of these structures has a certain degree of manufacturing adjustment to accommodate various anatomical variations, as well as differences related to the person's age.

The described device acts as an element that prevents the vibratory action of air during breathing and the structural relaxation of the area caused by decreased nerve impulse frequency, reduced muscle tone, or the person's postural actions. It also maintains a permanent air entry pathway, which can prevent future involuntary airway obstructions, interruptions in breathing, and chronic snoring that could lead to greater anatomical deterioration. Additionally, it helps prevent injuries to the mandibular joint, dental wear, or breakage of teeth.

From a technical standpoint, it is noteworthy that the device avoids postural impact, the use of masks, tubes, or other external devices requiring external power sources.

Furthermore, the device includes an internal signaling system that changes color on the front part, serving as a visible indicator of the patient's status and device operation. It also features a quick-access mouthpiece adapter for emergency airway access (such as during Cardiopulmonary Resuscitation, CPR) and allows for additional vital sign readings through integrated electronic sensors controlled by a computer application.

Thus, starting from the implant placed in the user's tongue and optionally at the palate, the device is applied as follows:
1. Extract the tongue until it is fixed to the prosthetic bridge implant via magnetic attraction.
2. Insert the device into the mouth, securing the teeth with the dental splint.
3. The movement dynamics of the palate cause the upper supports to automatically lock into place, although adaptation to the wearer during placement is anticipated.

Optionally, the device can be supplied with a QR code or link to access a control application via a mobile phone, which may include sensors for measuring apneas and vital signs relevant for remote monitoring or control, as well as other conditions.

The described device is adaptable to any user, preventing issues such as bruxism, sleep apnea, snoring, and other conditions. It is also suitable for assisted cardiopulmonary resuscitation and continuous monitoring with color-changing indicators to signal alarms.

1. Oral device for the treatment of sleep apnea and chronic snoring, which involves at least one ferrous implant intended to be implanted in the longitudinal position of the lower part of the tongue. It is characterized in that it includes a dental splint materialized as a main body (1) based on an arched vertical septum, which terminates superiorly and inferiorly in two prosthetic bridges: a lower (2) and an upper (3), adapted to the palate area and the sub-lingual area of the user. The main body (1) further ends at its top and bottom with respective sub-plates: an upper sub-plate (4) and a lower sub-plate (5), adapted to the user's dental arches. It has been foreseen that the lower prosthetic bridge (2) includes a complementary magnetic element (7) for interaction with the implant located in the lower tongue area. Meanwhile, the upper prosthetic bridge (3) features at its posterior end a flexible arm (8), which terminates in a pair of spherical terminals (9) that are adjustable due to the flexibility of the arm itself. The main body (1) also includes a wide front window (6) in its central zone, while on the sides of the arched frontal septum there are additional holes (10) and (11). From these holes extend tubes (12) into the oral cavity, serving as means for additional internal pressure relief during air circulation and for attaching accessory elements.

2. Oral device for the treatment of sleep apnea and chronic snoring, according to claim 1, characterized in that the pair of terminals (9) are magnetized, being magnetically complementary to additional ferrous implants that can be implanted at the palate level.

3. Oral device for the treatment of sleep apnea and chronic snoring, according to claim 1, characterized in that the front window (6) features an entry mouth with a tubular configuration.

4. Oral device for the treatment of sleep apnea and chronic snoring, according to claim 1, characterized in that it includes fixed and automatic color indicators for signaling varying degrees of condition severity.

5. Oral device for the treatment of sleep apnea and chronic snoring, according to claim 1, characterized in that it includes sensors such as airflow measurement sensors, heart rate sensors, oxygen saturation sensors, or others, as well as communication means with an external electronic device such as a mobile phone, tablet, or similar, equipped with analysis software for the obtained data.
